(19) United States
(12) Patent Application Publication
(10) Pub. No.: US 2021/0393974 A1
(43) Pub. Date: Dec. 23, 2021

(54) LED THERAPY MASK

(71) Applicant: Massachusetts Institute of Technology, Cambridge, MA (US)

(72) Inventors: Jeehwan Kim, Cambridge, MA (US); Hanwool Yeun, Cambridge, MA (US); Da Hye Park, Cambridge, MA (US)

(73) Assignee: Massachusetts Institute of Technology, Cambridge, MA (US)

(21) Appl. No.: 17/283,506

(22) PCT Filed: Oct. 15, 2019
(86) PCT No.: PCT/US19/56231

§ 371 Date: Apr. 7, 2021

Related U.S. Application Data
(60) Provisional application No. 62/745,864, filed on Oct. 15, 2018.

(51) Int. Cl. A61N 5/06

(52) U.S. Cl. CPC A61N 5/0601; A61N 5/0616; A61N 2005/0663; A61N 2005/0612; A61N 2005/0659; A61N 2005/0652

(57) ABSTRACT

LED therapy masks, and associated systems and methods, are generally described. In one example, a wearable system for skin treatment of a user includes a substrate and a plurality of pixels disposed on the substrate. In this example, each pixel in the plurality of pixels includes a stack of light emitting diodes (LEDs) including a first LED configured to emit first light at a first wavelength and a second LED configured to emit light at a second wavelength. In this example, each pixel also includes a first microneedle in optical communication with the first LED and configured to guide the first light into the skin of the user during use and a second microneedle in optical communication with the second LED and configured to guide the second light into the skin of the user during use.

### RELATED APPLICATIONS

This application claims priority under 35 U.S.C. § 119(e) to U.S. Provisional Application No. 62/745,864, filed Oct. 15, 2018, and entitled “Fully Efficient LED Therapy Mask,” which is incorporated herein by reference in its entirety for all purposes.

### TECHNICAL FIELD

LED therapy masks, and associated systems and methods, are generally described.

### BACKGROUND

Light emitting diode (LED) wearable masks have emerged as effective tools for long-term, real-time skin therapies. During these therapies, photons from LEDs are absorbed by chromophores and photo-acceptors, thereby promoting the metabolic activity of the cells. The penetration depth of the photons usually depends on the wavelength of the photons. Accordingly, photons at different wavelengths can be used to generate different clinical effects. For example, blue light (i.e., λ is about 400 nm) can significantly affect the epidermis and help acne treatment through bacterial removal. Red light (i.e., λ is about 700 nm) can reach the dermis and stimulate the production of collagen and elastin, thereby assisting the tightening of the skin.

One drawback of existing LED wearable masks is the undesired absorption of photons by the skin. For example, the outer-most barrier that protects humans is the epidermis, which can absorb light significantly. For example, the intensity of blue light and red light can decrease by 68% and 35%, respectively, after transmission through the epidermis. Therefore, existing on-skin light therapy masks typically have limited light therapy efficiency. In addition, the weight of a conventional mask is usually over 2 kg, thereby limiting its use for real-time treatment.

### SUMMARY

Embodiments of the present invention include apparatus, systems, and methods for skin therapy. Certain embodiments are related to LED therapy masks that are highly efficient. In some embodiments, the LED therapy mask comprises features that guide the light emitted by the LEDs into the skin with little or no absorption by the epidermis.

In one example, a wearable system for skin treatment of a user includes a substrate and a plurality of pixels disposed on the substrate. In this example, each pixel in the plurality of pixels includes a stack of light emitting diodes (LEDs) including a first LED configured to emit first light at a first wavelength and a second LED configured to emit second light at a second wavelength. Each pixel also includes, in this example, a first microneedle in optical communication with the first LED and configured to guide the first light into the skin of the user during use and a second microneedle in optical communication with the second LED and configured to guide the second light into the skin of the user during use.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. lA is a cross-sectional schematic diagram of skin.

FIG. 1B shows a schematic of a wearable system for skin therapy on the skin shown in FIG. 1A, in accordance with some embodiments.

FIG. 2 shows a schematic of a wearable system including an array of light emitting diodes (LEDs) for skin therapy, in accordance with some embodiments.

FIGS. 3A-3E illustrate a method of fabricating LEDs for a wearable system for skin therapy, in accordance with some embodiments.

FIGS. 4A-4D illustrate a method of fabricating vertically stacked LEDs, in accordance with some embodiments.

FIG. 5 shows a schematic of a vertically stacked LED device that can be fabricated via the method illustrated in FIGS. 4A-4D, in accordance with some embodiments.

FIGS. 6A-6C illustrate a method of fabricating microneedles on an LED array, in accordance with some embodiments.

### DETAILED DESCRIPTION

To address the drawbacks in conventional LED therapy masks, systems and methods described herein employ an approach in which microneedles are coupled to LED arrays so as to guide the light beams from the LEDs into the skin of the user. The guiding of the microneedles can significantly decrease the absorption of light by the epidermis, thereby increasing the light therapy efficiency. In addition, in accordance with certain embodiments, two-dimensional layer transfer (2DLT) techniques are employed to fabricate the wearable system. The 2DLT technique can fabricate compact and lightweight semiconductor devices, thereby reducing the weight of the resulting therapy system.

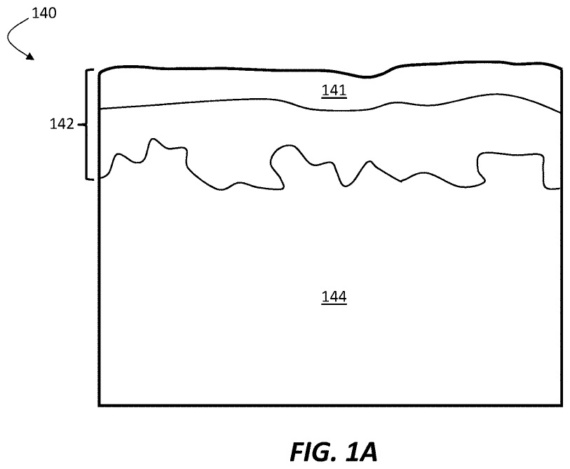

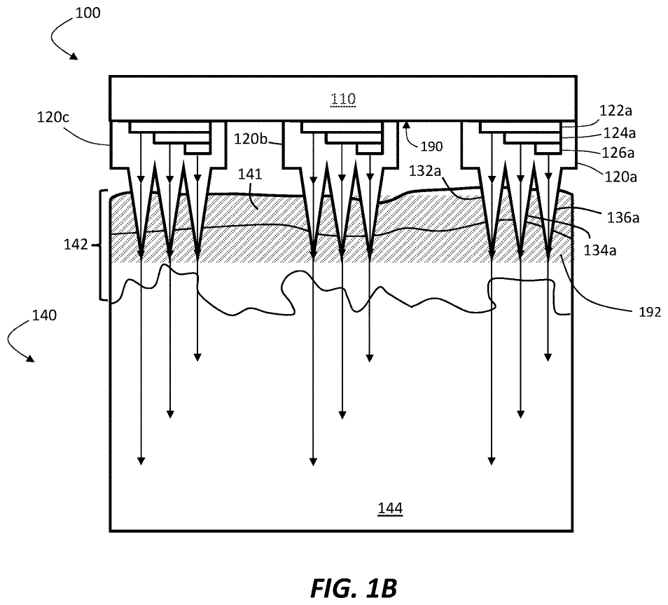

FIG. 1A is a cross-sectional schematic diagram of skin 140. In FIG. 1A, skin 140 comprises stratum corneum 141, epidermis 142, and dermis 144.

FIG. 1B shows a schematic of a wearable system 100 for skin therapy, in accordance with some embodiments. System 100 is configured to be applied to skin 140 of a user for skin therapy (or any other therapy that can benefit from light emitted by system 100). For example, in FIG. 1B, system 100 is shown in contact with skin 140 from FIG. 1A. System 100 includes, in accordance with certain embodiments, a substrate 110 and an array of pixels 120 (labeled 120a, 120b, and 120c in FIG. 1B) disposed on substrate 110. Three pixels are illustrated in FIG. 1B, but any other number of pixels can also be used. The description below uses one pixel 120a for illustrative purposes. In some embodiments, substrate 110 can include a flexible film that can be conformally applied to skin 140 of the user. In some embodiments, the thickness of the substrate can be substantially equal to or less than 50 μm (e.g., about 50 μm, about 45 μm, about 40 μm, about 35 μm, about 30 μm, or less, including any values and sub ranges in between). In some embodiments, substrate 110 can include a bio-compatible material, such as silicone.

Pixel 120a further includes, in accordance with certain embodiments, a first LED 122a operating at a first wavelength, a second LED 124a operating at a second wavelength, and a third LED 126a operating at a third wavelength. As would be understood by a person of ordinary skill in the art, the operational wavelength of a given LED is the wavelength the LED emits at the highest intensity. Although three LEDs are illustrated in FIG. 1B, any other number of LEDs can also be used. For example, each pixel can include only two LEDs. In another example, each pixel can include more than 3 LEDs.

In some embodiments, the first wavelength (the operational wavelength of the first LED) can be about 400 nm to about 600 nm (e.g., about 400 nm, about 450 nm, about 500 nm, about 550 nm, or about 600 nm, including any values and sub ranges in between). Light at these wavelengths can be used for bacteria removal and thereby can be used for acne treatment.

In some embodiments, the second wavelength (the operational wavelength of the second LED) can be about 600 nm to about 700 nm (e.g., about 600 nm, about 650 nm, or about 700 nm, including any values and sub ranges in between). Light at these wavelengths can be used to repair/rejuvenate collagens and therefore can be used for improving skin elasticity, aiding in wrinkle reduction, and helping maintain a more youthful appearance. In some embodiments, the second wavelength is at least 10 nm, at least 20 nm, or at least 50 nm different from (e.g., higher than) the first wavelength.

In some embodiments, the third wavelength (the operational wavelength of the third LED) can be about 700 nm to about 1000 nm (e.g., about 700 nm, about 750 nm, about 800 nm, about 850 nm, about 900 nm, about 950 nm, or about 1000 nm, including any values and sub ranges in between). Light at these wavelengths can be used for stimulation of mitochondria and ATP, thereby allowing the applications in wound healing and pain relief. In some embodiments, the third wavelength is at least 100 nm, at least 120 nm, or at least 150 nm different from (e.g., higher than) the first wavelength. In some embodiments, the third wavelength is at least 10 nm, at least 20 nm, or at least 50 nm different from (e.g., higher than) the second wavelength.

As illustrated in FIG. 1B, pixel 120a is coupled to an array of microneedles, including a first microneedle 132a, a second microneedle 134a, and a third microneedle 136a. First microneedle 132a is in optical communication with first LED 122a to guide the light from first LED 122a to skin 140. Second microneedle 134a is in optical communication with second LED 124a to guide the light from second LED 124a to skin 140. Third microneedle 136a is in optical communication with third LED 126a to guide the light from third LED 126a to the skin 140.

In some embodiments, microneedles 132a, 134a, and 136a are configured to penetrate into epidermis 142 of skin 140 such that the emitted light is guided into the dermis 144 of skin 140 so as to increase the therapy efficiency. Microneedles 132a, 134a, and 136a can be configured such that they do not penetrate into the dermis. In some embodiments, the length of the microneedles 132a, 134a, and 136a can be shorter than the distance from the skin surface to pain receptors in the skin (e.g., in the dermis) so as to allow painless therapy. In some embodiments, the length of the microneedles 132a, 134a, and 136a can be substantially equal to or less than about 200 μm (e.g., about 200 μm, about 180 μm, about 160 μm, about 140 μm, about 120 μm, or about 100 μm, including any values and sub ranges in between).

In some embodiments, the microneedles 132a, 134a, and 136a can have a tapered shape along their lengths (e.g., a conical shape). Use of microneedles that are tapered along their lengths can aid in focusing the light from the LEDs (e.g., 122a, 124a, and 126a, in FIG. 1B). In some embodiments, microneedles 132a, 134a, and 136a can have an inverse pyramid shape. In some embodiments, microneedles 132a, 134a, and 136a can have a cylindrical shape having a spherical tip to focus light.

The microneedles can be formed from any of a variety of suitable materials. In some embodiments, microneedles 132a, 134a, and 136a comprise a polymeric material (e.g., an organic polymer). In certain embodiments, microneedles 132a, 134a, and 136a comprise a hard polymer (e.g., an epoxy, such as SU-8).

Microneedles 132a, 134a, and 136a can, in some embodiments, create a pathway through the epidermis through which light may be efficiently transmitted (e.g., into the dermis). For example, in FIG. 1B, microneedles 132a, 134a, and 136a establish a pathway through region 192 (illustrated in FIG. 1B using cross-hatching) such that light from LEDs 122a, 124a, and 126a may reach dermis 144 more efficiently.

In some embodiments, the microneedles guide the light emitted by the LEDs into the skin with little or no absorption of the light by the epidermis. In some such embodiments, the microneedles provide a light penetration path such that the microneedles guide the light through the epidermis and into the dermis. In some embodiments, less than 20%, less than 10%, less than 5%, less than 2%, or less than 1% (or none) of the light emitted by the LEDs is absorbed by the epidermis.

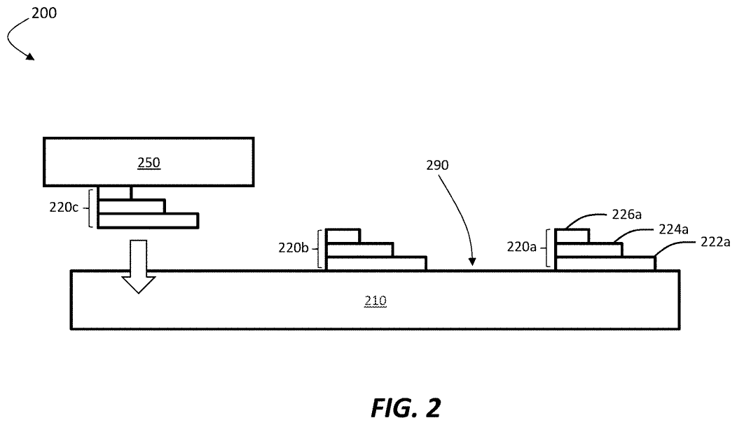

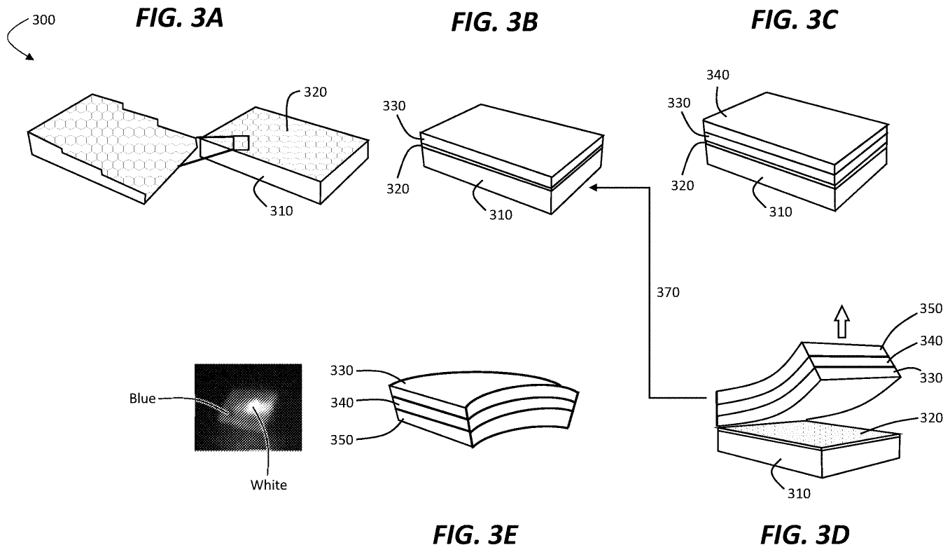

FIG. 2 shows a schematic of a wearable system 200 including an array of light emitting diodes (LEDs) for skin therapy, in accordance with some embodiments. System 200 includes a substrate 210 and an array of pixels 220a, 220b, and 220c disposed on substrate 210. Pixel 220a includes a stack of LEDs 222a, 224a, and 226a, each of which operates at a distinct wavelength. Substrate 210 can be flexible so as to allow conformal application onto the skin of the user. In addition, LEDs 220 can be fabricated via a 2DLT technique (see, e.g., FIG. 3) such that the entire system 200 can be lightweight (e.g., less than 500 g, less than 300 g, less than 200 g, less than 100 g, less than 50 g, less than 30 g, less than 20 g, less than 10 g, or less than 5 g, including any values and sub ranges in between). To this end, a stamp 250 can be employed to transfer the pixels (e.g., 220c in FIG. 2) from another substrate (e.g., the growth substrate for the LEDs) onto substrate 210. More details of an example fabrication process are provided below with reference to FIGS. 3-5.

FIGS. 3A-3E illustrate a method 300 of fabricating LEDs for a wearable system for skin therapy, in accordance with some embodiments. In this method, a release layer 320 (e.g., a two-dimensional material, such as graphene) is fabricated on a growth substrate 310 (e.g., SiC), as shown in FIG. 3A. As one example, graphene can be formed on a SiC substrate via graphitization of the SiC. In some embodiments, the release layer can be a single layer of graphene (e.g., a single layer of crystalline graphene). In certain embodiments, the release layer can be continuous across its lateral dimensions.

In FIG. 3B, device layer 330 (e.g., GaN or GaAs) has been fabricated on release layer 320. The device layer may be formed, for example, via epitaxial growth on the release layer. As one example, GaN can be epitaxially grown on graphene located on a SiC substrate. In some embodiments, the device layer can be seeded by the underlying substrate and/or by the release layer. In some embodiments, the seeding of device layer 330 by growth substrate 310 can occur even when there is not direct contact between device layer 330 and growth substrate 310. For example, in accordance with certain embodiments, growth substrate 310 may have a potential field (e.g., created by van der Waals forces and/or other atomic or molecular forces) and release layer 320 may be so thin (e.g., in the case of a monolayer of continuous graphene) that the potential field of growth substrate 310 reaches beyond release layer 320 and interacts with the region within which device layer 330 is formed. As a result, in some embodiments, the potential field from growth substrate 310 affects the growth of device layer 330. In some such embodiments, the crystallographic orientations of growth substrate 310 and device layer 330 can be matched.

As shown in FIG. 3C, in certain embodiments, a stressor 340 (e.g., a nickel stressor) is then fabricated on device layer 330. In some embodiments, as shown in FIG. 3D, a tape 350 is formed on stressor 340. Tape 350 is employed, in accordance with certain embodiments, to remove device layer 330 from release layer 320, leaving a platform including the substrate 310 and the release layer 320 for one or more reuses (as indicated by arrow 370 linked FIG. 3D and FIG. 3B). The removed device layer 330 (shown in FIG. 3E) can then be transferred to another substrate (e.g., a flexible film) to construct a wearable system for skin therapy.

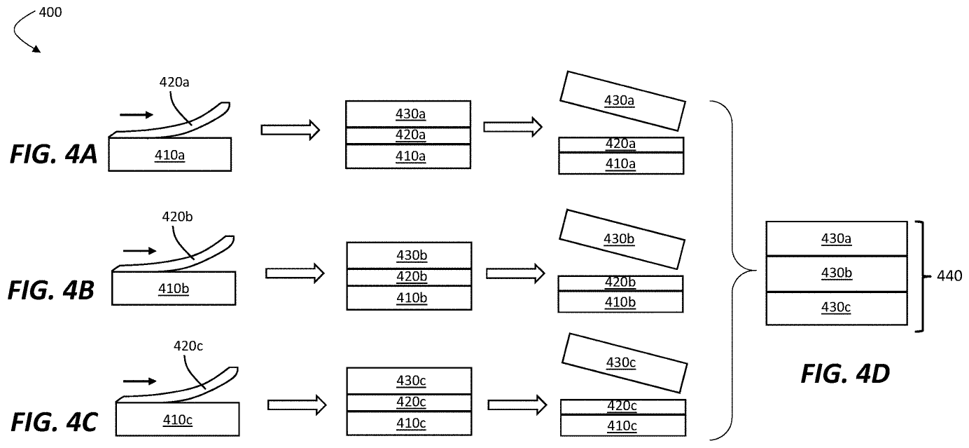

FIGS. 4A-4D illustrate a method 400 of fabricating vertically stacked LED devices. As shown in FIG. 4A, in this method 400, in accordance with certain embodiments, a first release layer 420a (e.g., a 2D material, such as graphene) is formed on a first substrate 410a, then a first LED layer 430a is formed on the first release layer 420a. The fabricated LED layer 430a is then removed from first release layer 420a, leaving a platform including first substrate 410a and first release layer 420a for the next cycle of fabrication, as illustrated in FIG. 4A. First LED layer 430a includes a first crystalline inorganic semiconductor configured to emit light at a first wavelength (e.g., a wavelength of blue light).

Similarly, in accordance with certain embodiments, FIG. 4B shows that a second release layer 420b (e.g., a 2D material, such as graphene) is formed on a second substrate 410b, then a second LED layer 430b is formed on second release layer 420b. The fabricated LED layer 430b is then removed from second release layer 420b, leaving a platform including second substrate 410b and second release layer 420b for the next cycle of fabrication. Second LED layer 430b includes a second crystalline inorganic semiconductor configured to emit light at a second wavelength (e.g., a wavelength of green light).

FIG. 4C shows, in accordance with some embodiments, that a third release layer 420c (e.g., a 2D material, such as graphene) is formed on a third substrate 410c, followed by forming a third LED layer 430c on third release layer 420c. The fabricated LED layer 430c is then removed from third release layer 420c, leaving a platform including the third substrate 410c and third release layer 420c for the next cycle of fabrication. The third LED layer 430c includes a third crystalline inorganic semiconductor configured to emit light at a third wavelength (e.g., a wavelength of red light).

After being removed from corresponding release layers 420a, 420b, and 420c (collectively referred to as release layers 420), LED layers 430a, 430b, and 430c (collectively referred to as LED layers 430) are stacked together to form a vertically stacked LED device 440, as shown in FIG. 4D. Since each LED layer 430a, 430b, and 430c is configured to emit light at a distinct wavelength (e.g., wavelengths corresponding to red, green, and blue light, respectively), the LED device 440 can emit light at any color by adjusting the amount of light emitted from each LED layer 430a, 430b, and 430c. FIG. 4D shows that second LED layer 430b is sandwiched between first LED layer 430a and third LED layer 430c. Other configurations can also be used. For example, first LED layer 430a can be disposed between second LED layer 430b and third LED layer 430c.

In addition, device 440 may have only two LED layers, four LED layers, five LED layers, or any other number of LED layers. For example, in some embodiments, device 440 can include two LED layers: one configured to emit yellow light and the other configured to emit blue light. In another example, device 440 can include four LED layers configured to emit red, green, blue, and yellow light, respectively. The thickness of each LED layer 430 can be about 1 μm to about 100 μm (e.g., about 1 μm, about 2 μm, about 3 μm, about 5 μm, about 10 μm, about 20 μm, about 30 μm, about 50 μm, or about 100 μm, including any values and sub ranges in between).

In one example, device 440 can be further patterned into multiple pixels after

LED layers 430a, 430b, and 430c are stacked as shown in FIG. 4D. In another example, each LED layer 430a, 430b, and 430c can be pre-patterned before they are stacked together to form LED device 440.

The first wavelength of light emitted by first LED layer 430a can be, in some embodiments, anywhere from about 360 nm to about 490 nm, or anywhere from about 400 nm to about 600 nm. In some embodiments, the first crystalline inorganic semiconductor can include, for example, gallium nitride (GaN), zinc selenide (ZnSe), indium gallium nitride (InGaN), or silicon carbide (SiC).

The second wavelength of light emitted by the second LED layer 430b can be, in some embodiments, about 490 nm to about 580 nm, or about 600 nm to about 700 nm.

The corresponding second crystalline inorganic semiconductor can include, for example, gallium(III) phosphide (GaP), aluminum gallium indium phosphide (AlGaInP), aluminum gallium phosphide (AlGaP), or indium gallium nitride (InGaN)/Gallium(III) nitride (GaN).

The third wavelength of light emitted by the third LED layer 430c can be, in some embodiments, anywhere from about 580 nm to about 760 nm, or anywhere from about 700 nm to about 1000 nm. In certain embodiments, the third crystalline inorganic semiconductor can include, for example, aluminum gallium arsenide (AlGaAs), gallium arsenide phosphide (GaAsP), aluminum gallium indium phosphide (AlGaInP), or gallium(III) phosphide (GaP).

Substrates 410a, 410b, and 410c (collectively referred to as LED growth substrates 410) can include the same semiconductor material as used in the respective LED layers 430a, 430b, and 430c. For example, first substrate 410a and first LED layer 430a can each be formed of the same crystalline inorganic semiconductor material. If release layers 420 are thin enough (e.g., about 1 nm to about 10 nm), this configuration allows lattice matching between the LED growth substrates 410 and the LED layers 430. Therefore, fabricated LED layers 430 can have high crystalline quality. For example, the density of defects, such as dislocations, can be on the order of about 10⁴/cm²-10⁸/cm². Alternatively, LED growth substrates 410a/b/c may use a different material from the material of the corresponding LED layer 430a/b/c, in which case the growth of the LED layers 430 can be seeded by release layers 420. More information about different seeding schemes can be found in PCT Application No. PCT/US2016/050701, filed Sep. 6, 2016, published as International Patent Application Publication No. WO 2017/044577 on Mar. 16, 2017, and entitled “SYSTEMS AND METHODS FOR GRAPHENE BASED LAYER TRANSFER,” which is hereby incorporated by reference in its entirety.

Release layers 420 include, in accordance with certain embodiments, a two-dimensional (2D) material to facilitate the transfer of fabricated LED layers 430 from LED growth substrates 410 to a host substrate (not shown in FIGS. 4A-4D) for stacking. Various types of 2D materials can be used for release layers 420. In one example, release layers 420 include graphene (e.g., monolayer graphene or multilayer graphene). In another example, release layers 420 include transition metal dichalcogenide (TMD) monolayers, which are atomically thin semiconductors of the type MX₂, with M being a transition metal atom (e.g., Mo, W, etc.) and X being a chalcogen atom (e.g., S, Se, or Te). In a TMD lattice, one layer of M atoms is usually sandwiched between two layers of X atoms. In yet another example, release layers 420 can include a single-atom layer of metal, such as palladium and rhodium. The three release layers 420 can include the same material or different materials, depending in part on the materials of LED layers 430.

Out of these 2D materials, graphene can have several desirable properties. For example, graphene is a crystalline film and is a suitable substrate for growing epitaxial over-layers. Second, graphene's weak interaction with other materials can substantially relax the lattice mismatching rule for epitaxial growth, potentially permitting the growth of most semiconducting films with low defect densities. Third, epilayers grown on a graphene substrate can be easily and precisely released from the substrate owing to graphene's weak van der Waals interactions, thereby allowing rapid mechanical release of epilayers without post-release reconditioning of the released surface. Fourth, graphene's mechanical robustness can increase or maximize its reusability for multiple growth/release cycles.

A release layer 420 including graphene is also referred to as a graphene layer 420 herein. In one example, a graphene layer 420 can be grown directly on LED growth substrate 410. In another example, a graphene layer 420 can be grown on a separate substrate (also referred to as a graphene growth substrate) and then transferred to LED growth substrate 410.

A graphene layer 420 can be fabricated on a separate substrate via various methods. In one example, the graphene layer 420 can include an epitaxial graphene with a single-crystalline orientation and the graphene growth substrate can include a (0001) 4H-SiC wafer with a silicon surface. The fabrication of a graphene layer 420 can include multiple annealing steps. A first annealing step can be performed in H₂ gas for surface etching, and a second annealing step can be performed in Ar for graphitization at high temperature (e.g., about 1,575° C.).

In another example, the graphene layer 420 can be grown on the graphene growth substrate via a chemical vapor deposition (CVD) process. The graphene growth substrate can include a nickel substrate or a copper substrate. Alternatively, the graphene growth substrate can include an insulating substrate of SiO₂, HfO₂, Al₂O₃, Si₃N₄, or practically any other planar material compatible with high temperature CVD.

In yet another example, the graphene growth substrate can be any substrate that can hold a graphene layer 420, and the fabrication can include a mechanical exfoliation process. In this example, the graphene growth substrate can function as a temporary holder for each graphene layer 420.

Various methods can also be used to transfer graphene layers 420 from the graphene growth substrate to LED growth substrates 410. In one example, a carrier film can be attached to a given graphene layer 420. The carrier film can include a thick film of Poly(methyl methacrylate) (PMMA) or a thermal release tape and the attachment can be achieved via a spin-coating process. After the combination of the carrier film and graphene layer 420 is disposed on LED growth substrate 410, the carrier film can be dissolved (e.g., in acetone) for further fabrication of one or more LED layers 430 on graphene layer 420.

In another example, a stamp layer including an elastomeric material, such as polydimethylsiloxane (PDMS), can be attached to graphene layer 420 and the graphene growth substrate can be etched away, leaving the combination of the stamp layer and graphene layer 420. After the stamp layer and graphene layer 420 are placed on LED growth substrate 410, the stamp layer can be removed by mechanical detachment, producing a clean surface of graphene layer 420 for further processing.

In yet another example, a self-release transfer method can be used to transfer a graphene layer 420 to a corresponding LED growth substrate 410. In this method, a self-release layer is first spin-cast over graphene layer 420. An elastomeric stamp is then placed in conformal contact with the self-release layer. The graphene growth substrate can be etched away to leave the combination of the stamp layer, the self-release layer, and graphene layer 420. After this combination is placed on corresponding LED growth substrate 410, the stamp layer can be removed mechanically and the self-release layer can be dissolved under mild conditions in a suitable solvent. The release layer can include polystyrene (PS), poly(isobutylene) (PIB) and Teflon AF (poly[4,5-difluoro-2,2-bis(trifluoromethyl)-1,3-dioxole-co-tetrafluoroethylene]).

In some examples, release layers 420 can be porous. A porous release layer 420 can be fabricated by patterning a 2D material layer. For example, a porous film (e.g., oxide, nitride, or photoresist film) can be disposed on an intact 2D material layer. The porous film can have a high density of pinholes (e.g., about one hole per square micron). Dry etching using Ar plasma or O₂ plasma can be then carried out to open up the pinholes, thereby allowing ions in the etching plasma to propagate through the porous film and arrive at the 2D material layer. The etching plasma then etches the portion of the 2D material layer directly underneath the pinholes in the porous film to create a porous release layer. The porous film can then be removed, leaving the porous release layer for further processing (e.g., growth of LED layers 430). In one example, the porous film includes photoresist material and can be removed by acetone. In another example, the porous film includes oxide or nitride and can be removed by hydrogen fluoride (HF).

The fabrication of the LED layers 430 can be carried out via epitaxial growth using any of a variety of appropriate semiconductor fabrication techniques known in the art. For example, low-pressure Metal-Organic Chemical Vapor Deposition (MOCVD) can be used to grow LED layers 430 including GaN on release layers 420, which in turn is disposed on growth substrates 410. In this example, release layers 420 and growth substrates 410 can be baked (e.g., under H₂ for >15 min at >1,100° C.) to clean the surface. Then the deposition of LED layers 430 including GaN can be performed at, for example, 200 mbar. Trimethylgallium, ammonia, and hydrogen can be used as the Ga source, nitrogen source, and carrier gas, respectively. A modified two-step growth can be employed to obtain flat GaN epitaxial films on release layers 420. The first step can be carried out at a growth temperature of 1,100° C. for a few minutes where guided nucleation at terrace edges can be promoted. The second growth step can be carried out at an elevated temperature of 1,250° C. to promote lateral growth. Vertical GaN growth rate in this case can be around 20 nm per min.

In one example, the lattices of growth substrates 410 are matched to their corresponding LED layers 430, in which case growth substrates 410 function as seeds for the growth of LED layers 430. For example, the epitaxial layer and the substrate can include the same semiconductor material. In these instances, release layers 420 can be porous or thin enough (e.g., a single layer, or monolayer, of graphene). Sandwiching release layers 420 between growth substrates 410 and LED layers 430 can facilitate quick and damage-free release and transfer of LED layers 430.

In another example, a given release layer 420 can be thick enough (e.g., several layers of graphene) to function as a seed for growing the corresponding LED layer 430, in which case LED layers 430 can be latticed-matched to release layers 420. In yet another example, growth substrates 410 together with release layers 420 can function as the seeds to grow LED layers 430.

Using graphene in a release layer 420 as a seed to fabricate a corresponding LED layer 430 can also increase the tolerance to mismatch between the lattice constants of the LED material and graphene. Without being bound by any particular theory or mode of operation, surfaces of two-dimensional (2D) materials (e.g., graphene) or quasi-2D layered crystals typically have no dangling bonds and interact with adjacent materials via weak van der Waals like forces. Due to the weak interaction, an epilayer can grow from the beginning with its own lattice constant forming an interface with a small amount of defects. This kind of growth is referred to as Van Der Waals Epitaxy (VDWE). The lattice matching condition can be drastically relaxed for VDWE, allowing a large variety of different heterostructures even for highly lattice mismatched systems. In practice, the lattice mismatch can be about 0% to about 70% (e.g., about 0%, about 10%, about 20%, about 30%, about 40%, about 50%, about 60%, and about 70%, including any values and sub-ranges in between).

LED layers 430 can be transferred to the host substrate using a stressor layer. For example, a stressor layer (e.g., a high-stress metal film, such as Ni film) can be formed on a given LED layer 430, followed by formation of a tape layer on the stressor layer. The tape layer and the stressor layer can be used to mechanically exfoliate LED layers 430 from release layers 420 at a fast release rate by applying high strain energy to the interface between LED layers 430 and release layers 420. Without being bound by any particular theory, the release rate can be fast at least due to the weak van der Waals bonding between graphene and other materials such as LED layers 430.

Once LED layers 430 are placed on the host substrate, the tape layer and the stressor layer can be removed, leaving LED layers 430 for further processing, such as formation of metal contacts. In some examples, the tape layer and the stressor layer can be etched away by a FeCl₃-based solution.

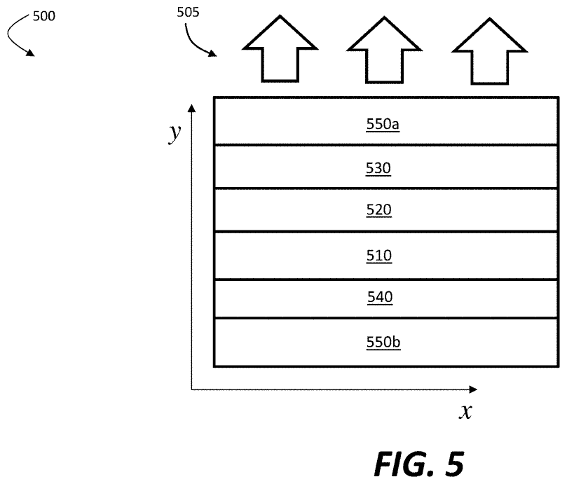

FIG. 5 shows a schematic of a vertically stacked LED device 500 that can be fabricated via method 400 illustrated in FIGS. 4A-4D. Device 500 can be used in the pixels for wearable systems that can be used for skin therapy (e.g., in pixels 120 in wearable system 100 of FIG. 1B). Device 500 includes three LED layers 510 (e.g., a red LED layer), 520 (e.g., a green LED layer), and 530 (e.g., a blue LED layer) vertically stacked to form an LED stack.

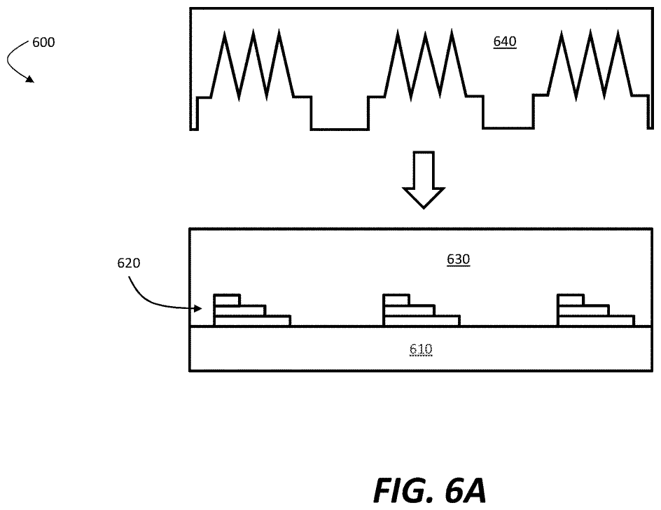

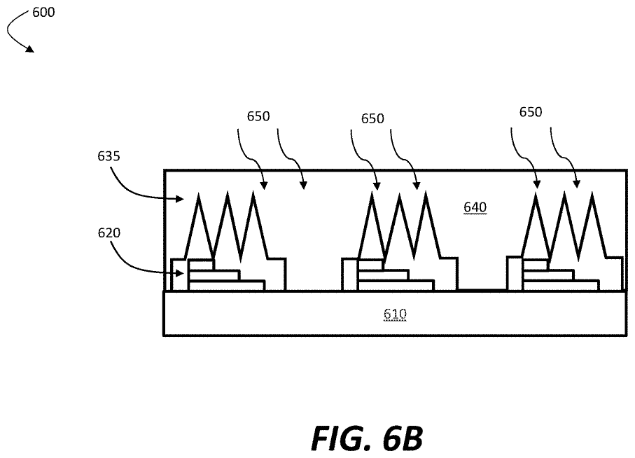

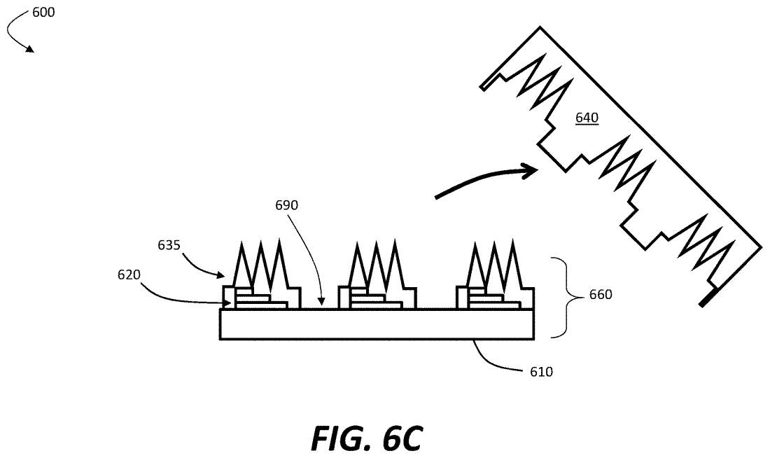

In certain embodiments, the LEDs (e.g., the first LED, the second LED, the optional third LED, and optional additional LEDs) can be stacked along a first direction substantially perpendicular to (e.g., within 15°, within 10°, within 5°, or within 2° of perpendicular to) the skin of the user during use. For example, in some embodiments, the y-axis in FIG. 5 (and/or equivalent stack directions for the LED stacks in FIG. 1B, FIG. 2, and/or FIG. 6C) is substantially perpendicular to the skin of the user during use. In some embodiments, the LEDs (e.g., the first LED, the second LED, the optional third LED, and optional additional LEDs) are disposed on a plane substantially parallel to (e.g., within 15°, within 10°, within 5°, or within 2° of parallel to) the skin of the user during use. For example, in some embodiments, the x-axis of FIG. 5 (and/or surface 190 of substrate 110 in FIG. 1B, and/or surface 290 of substrate 210 in FIG. 2, and/or surface 690 of substrate 610 in FIG. 6C) is substantially parallel to the skin of the user during use.

In accordance with certain embodiments, first LED layer 510 is configured to emit light at a first wavelength (e.g., about 580 nm to about 760 nm), second LED layer 520 is configured to emit light at a second wavelength (e.g., about 490 nm to about 580 nm), and third LED layer 530 is configured to emit light at a third wavelength (e.g., about 390 nm to about 490 nm). In certain embodiments, light emitted by three LED layers 510, 520, and 530 is multiplexed to form output light 505 propagating along the optical axis of the device 500 (i.e., along the y direction as illustrated in FIG. 5). Device 500 also includes, in some embodiments, a reflector 540 (e.g., a nickel reflector) disposed on one end of the LED stack (e.g., coupled to first LED layer 510) so as to cause device 500 to emit light along only one direction (e.g., the y direction as in FIG. 5).

Two encapsulation layers 550a and 550b are used to encapsulate the LED stack and the reflector 540, in accordance with certain embodiments. In one example, two separate layers can be used as the encapsulation layers 550a and 550b. In another example, the two encapsulation layers 550a and 550b can be part of a single encapsulation package that substantially encloses the LED stack and the reflector 540.

In one example, the resulting LED lighting device can include multiple components, each of which is similar to device 500, to form a lighting array. Different components in the array can be configured to emit light at different wavelengths. In another example, the LED lighting device can include only one component like device 500. In this instance, the lateral dimension of device 500 (e.g., the dimension along the x direction as illustrated in FIG. 5) can be about 1 mm or greater (e.g., about 1 mm, about 2 mm, about 5 mm, about 10 mm, about 20 mm, or greater, including any values and sub ranges in between). More details about vertically stacked LED devices can be found in PCT Patent Application No. PCT/US2018/019392, filed Feb. 23, 2018, published as International Patent Application Publication No. WO 2018/156876 on Aug. 30, 2018, and entitled “METHODS AND APPARATUS FOR VERTICALLY STACKED MULTICOLOR LIGHT-EMITTING DIODE (LED) DISPLAY,” which is incorporated herein by reference in its entirety.

FIGS. 6A-6C illustrate a method 600 of fabricating microneedles on an LED array, in accordance with some embodiments. In the method 600, a polymer 630 is disposed on an LED array 620 that is disposed on a substrate 610, as illustrated in FIG. 6A. The LED array 620 can be the same as or substantially similar to the array of LEDs 120 shown in FIG. 1B and/or the array of LEDs 220 shown in FIG. 2 and described above. Polymer 630 can include, for example, a UV curable polymer, such as an SU-8 polymer. In FIG. 6A, polymer 630 is configured to cover the entire LED array 620. A mold 640 is then disposed on polymer 630. Mold 640 can include a UV transparent material (e.g., PDMS). In addition, mold 640 includes a depressed region having a shape of a microneedle array.

FIG. 6B shows that mold 640 is pressed against polymer 630 so as to define a microneedle array 635 made of the polymer material. In some embodiments, an alignment key can be defined on mold 640 so as to ensure that microneedle array 635 is formed on LED array 620 (i.e., aligned with the LED array 620). FIG. 6B also shows that a curing agent 650 (e.g., radiation, such as UV light) is applied over polymer 630 so as to cure polymer 630 and solidify microneedles 635.

FIG. 6C shows that after microneedles 635 are cured, mold 640 is removed from LED array 620, thereby forming device 660 including microneedles 635 disposed on LED arrays 620. Device 660 can be the same as or substantially similar to system 100 shown in FIG. 1B and described above. In some embodiments, the removal of mold 640 can be facilitated by an anti-sticking layer (also referred to as a non-stick layer) coated on the inner surface of mold 640. In some embodiments, the anti-sticking layer can include a self-assembled monolayer.

In some embodiments, the wearable systems described herein can be configured to use (or used) by human users. In certain embodiments, the wearable systems described herein can configured for use (or used) by non-human users (e.g., non-human animals).

While embodiments in which the microneedles do not penetrate into the dermis have been primarily described (and can be particularly advantageous for a number of reasons), the embodiments described herein are not necessarily so limited, and in other embodiments, the microneedles can penetrate through the epidermis of the user and reach a dermis of the user.

U.S. Provisional Application No. 62/745,864, filed Oct. 15, 2018, and entitled “Fully Efficient LED Therapy Mask” is incorporated herein by reference in its entirety for all purposes.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which examples have been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A wearable system for skin treatment of a user, the system comprising:
a substrate;
a plurality of pixels disposed on the substrate, each pixel in the plurality of pixels comprising:
a stack of light emitting diodes (LEDs) including a first LED configured to emit first light at a first wavelength and a second LED configured to emit second light at a second wavelength;
a first microneedle in optical communication with the first LED and configured to guide the first light into a skin of the user during use; and
a second microneedle in optical communication with the second LED and configured to guide the second light into the skin of the user during use.

2. The wearable system of claim 1, wherein the substrate comprises a flexible film having a thickness substantially equal to or less than 50 μm.

3. The wearable system of claim 1, wherein the substrate comprises a bio-compatible material.

4. The wearable system of claim 3, wherein the bio-compatible material comprises silicone.

5. The wearable system of claim 1, wherein the stack of LEDs comprises GaAs LEDs.

6. The wearable system of claim 1, wherein the first microneedle has a tapered structure.

7. The wearable system of claim 6, wherein the first microneedle has a conical structure.

8. The wearable system of claim 1, wherein the first microneedle has a structure that focuses the first light.

9. The wearable system of claim 1, wherein the first microneedle has a length substantially equal to or less than 200 μm.

10. The wearable system of claim 1, wherein the first microneedle is configured to penetrate into an epidermis of the user and emit light that reaches a dermis of the user.

11. The wearable system of claim 1, wherein the first microneedle comprises a curable polymer.

12. The wearable system of claim 11, wherein the curable polymer comprises an epoxy.

13. The wearable system of claim 1, wherein the stack of LEDs is stacked along a first direction substantially perpendicular to the skin of the user during use, and the first LED and the second LED are disposed on a plane substantially parallel to the skin of the user during use.

14. The wearable system of claim 1, wherein the first wavelength is about 400 nm to about 600 nm.

15. The wearable system of claim 1, wherein the second wavelength is about 600 nm to about 700 nm.

16. The wearable system of claim 1, wherein each pixel in the plurality of pixels further comprises:
a third LED configured to emit third light at a third wavelength; and
a third microneedle in optical communication with the third LED and configured to guide the third light into the skin of the user during use.

17. The wearable system of claim 16, wherein the third wavelength is about 700 nm to about 1000 nm.
